(19) United States
(12) Patent Application Publication
(10) Pub. No.: US 2006/0280645 A1
(43) Pub. Date: Dec. 14, 2006

(54) Method for terminal sterilization of transdermal delivery devices

(76) Inventors: Scott Sellers, San Mateo, CA (US); Yuh-Fun Maa, Millbrae, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/446,531

(22) Filed: Jun. 1, 2006

Related U.S. Application Data
(60) Provisional application No. 60/687,635, filed on Jun. 2, 2005.

(51) Int. Cl. A61F 13/02; A61L 2/00

(52) U.S. Cl. 422/22; 424/448

(57) ABSTRACT

A method and system for providing a terminally sterilized transdermal natriuretic peptide delivery device. A microprojection member having a plurality of stratum corneum-piercing microprojections is coated with a natriuretic peptide- formulation an exposed to sufficient radiation to sterilize the microprojection member while retaining sufficient activity of the natriuretic peptide. Preferably, the microprojection member is sealed in packing with an inert atmosphere and reduced moisture. The sterilizing radiation can be gamma radiation or e-beam, preferably delivered in a dose in the range of approximately 14-21 kGy. Also preferably, the irradiation is performed at −78.5-25° C. In preferred embodiments, the radiation is delivered at a rate greater than 3.0 kGy/hr.

### CROSS-REFERENCE TO RELATED APPLICATIONS

This application claims the benefit of U.S. Provisional Application No. 60/687,635, filed Jun. 2, 2005.

### FIELD OF THE PRESENT INVENTION

The present invention relates generally to transdermal agent delivery systems and methods. More particularly, the invention relates to methods for sterilizing a transdermal device adapted to deliver a natriuretic peptide.

### BACKGROUND OF THE INVENTION

It is well known that acute heart failure is the single most common cause of hospitalization in the United States for patents 65 years of age and older. Indeed, acute heart failure results in approximately one million hospitalizations each year.

Nesiritide, a recombinant form of human B-type natriuretic peptide (hBNP), is often used to treat patients with acute congestive heart failure who have dyspnea (i.e., shortness of breath) at rest or with minimal activity. The noted peptide, hBNP, is a naturally occurring protein that is secreted by the heart in response to acute heart failure, e.g., when the heart is unable to pump blood efficiently, hBNP is produced.

Details of the natriuretic peptide hBNP and other brain natriuretic peptides (BNPs) and recombinant techniques for production of the same are set forth in U.S. Pat. Nos. 5,114,923 and 5,674,710. The noted patents are expressly incorporated herein in their entirety.

Recent studies indicate that hBNP provides a number of additional physiologic (or therapeutic) effects, such as relaxation of blood vessels, (i.e., vasodilation), enhancing the excretion of sodium (i.e., natriuresis) and fluid (i.e., diuresis) and decreasing neurohormones (i.e, endothelin, aldosterone, angiutensin II). All of the noted physiologic effects (or actions) work in concert on the vessels, heart and kidney to decrease the fluid load on the heart, which improves cardiac performance.

Recent studies have also demonstrated a role for BNP in blocking TGF-B medicated cardiac fibroblast proliferation and myocardial fibrosis. Additional evidence further suggests an ability to inhibit cardiac remodeling after myocardial infarction.

Nesiritide's diuretic and potentially anti-fibrotic effects have also led to significant interest in its potential to address acute and chronic kidney disease. Historical exploration of BNP has demonstrated a potential-long term benefit from chronic administration in slowing disease progression towards ESRD and dialysis reliance.

At present, nesiritide is only administered via intravenous infusion. However, the direct injection of an active agent, such as hBNP, into the bloodstream is a difficult, inconvenient, painful and uncomfortable procedure that sometimes results in poor patient compliance.

Transdermal delivery is thus a viable alternative for administering active agents, particularly, nesiritide, that would otherwise need to be delivered via hypodermic injection or intravenous infusion. The word “transdermal”, as used herein, is a generic term that refers to delivery of an active agent (e.g., a therapeutic agent, such as a human brain natriuretic peptide or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery thus includes intracutaneous, intradermal and intraepidermal delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves mechanically penetrating the outermost skin layer(s) to create micropathways in the skin. There have been many techniques and devices developed to mechanically penetrate or disrupt the outermost skin layers to create pathways into the skin. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25 -400 microns and a microprojection thickness of only about 5 -50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is also possible to have the active agent that is to be delivered coated on the microprojections instead of contained in a physical reservoir. This eliminates the necessity of a separate physical reservoir and developing an agent formulation or composition specifically for the reservoir.

As stated, nesiritide is at present delivered solely via intravenous routes. It would thus be desirable to provide an agent delivery system that facilitates transdermal administration of nesiritide as well as other natriuretic peptides.

Parenteral pharmaceutical products, such as Natrecor®, must meet stringent standards of sterility. One conventional method for assuring a sterile product is aseptic manufacturing. However, the demands of maintaining a sterile environment throughout the manufacturing process are time-consuming, laborious, and extremely expensive.

A potentially attractive alternative to aseptic manufacturing is to sterilize the product at the end of the manufacturing process. Terminal sterilization is used routinely for stable small molecules. Unfortunately, this method presents major challenges for more labile biopharmaceutical products. In particular, complex biological molecular structures such as nesiritide must be protected from degradation to retain therapeutic activity.

In U.S. Pat. Nos. 6,346,216 and 6,171,549, Kent discloses the use of low irradiation rates for the sterilization of various biological molecules. However, these teachings fail to address specific conditions tailored for natriuretic peptides or for transdermal delivery devices. Kent also fails to provide any discussion regarding the effect of packaging on the product's stability and focuses on irradiation at room temperature.

It is therefore an object of the present invention to provide a method for conveniently sterilizing a transdermal device adapted to deliver a natriuretic peptide.

It is yet another object of the present invention to provide a method for sterilizing a transdermal delivery system that is more cost efficient than aseptic manufacturing.

Another object of the present invention is to provide a method for terminal sterilization of a natriuretic peptide adapted for transdermal delivery.

It is another object of the present invention to provide packaging conditions for a during sterilization.

Yet another object of the invention is to terminally sterilize a transdermal device for delivering a natriuretic peptide so that the peptide retains a substantial degree of activity.

### SUMMARY OF THE INVENTION

In accordance with the above objects and those that will be mentioned and will become apparent below, the method and system for terminally sterilizing a transdermal natriuretic peptide delivery device comprises the steps of providing a microprojection member and exposing the microprojection member to radiation selected from the group consisting of gamma radiation and e-beam, wherein the radiation is sufficient to reach a desired sterility assurance level. The microprojection member includes a plurality of stratum corneum-piercing microprojections with a biocompatible coating having at least one natriuretic peptide disposed thereon. Preferably, the microprojection member is sealed within packaging adapted to control environmental conditions surrounding the microprojection member. In one embodiment, the packing comprises a foil pouch.

In one aspect of the invention, sealing a desiccant inside the packaging reduces moisture within the packaging. Alternatively, the microprojection member is mounted on a pre-dried retainer ring prior to sealing the microprojection member inside the packaging. In a preferred embodiment, both a desiccant and a pre-dried retainer ring are used to reduce moisture within the sealed packaging.

In a further embodiment of the invention, the packaging is purged with an inert gas prior to sealing the microprojection member. Preferably, the packaging is purged with dry nitrogen.

The invention also comprises reducing the degradation of the natriuretic peptide during sterilization by adjusting the temperature at which the irradiation occurs. In one embodiment, the microprojection member is irradiated at a temperature in the range of approximately −78.5 to 25° C. The microprojection members can be irradiated at a temperature of −78.5° C. under dry ice conditions. In another embodiment, the microprojection member is irradiated at a temperature in the range of approximately 0-25° C. In another embodiment, the microprojection member is irradiated at an ambient temperature in the range of approximately 20-25° C.

According to the invention, the microprojection member receives a dose of radiation that is approximately 14 kGy. In another embodiment, the dose is approximately 16.5 kGy. In yet another embodiment, the dose is approximately 21 kGy.

In another embodiment, the invention includes exposing the microprojection member to radiation at a rate of greater than approximately 3.0 kGy/hr.

In further embodiments of the invention, the microprojection member is exposed to sufficient radiation to achieve a sterility assurance level of 10⁻⁶.

In other embodiments of the invention, an antioxidant is added to the coating formulation. Suitable antioxidants include methionine and ascorbic acid.

The methods of the invention also comprise sterilizing the microprojection member so that the natriuretic peptide retains at least approximately 95% of initial purity. More preferably, the natriuretic peptide retains at least approximately 98% of initial purity.

In a currently preferred embodiment of the invention, the method for terminally sterilizing a transdermal natriuretic peptide delivery device comprises the steps of providing a microprojection member, mounting the microprojection member on a pre-dried retainer ring, sealing the microprojection member inside packaging purged with nitrogen and adapted to control environmental conditions surrounding the microprojection member, and exposing the microprojection member to e-beam radiation, wherein the radiation is sufficient to reach a desired sterility assurance level. The microprojection member preferably includes a plurality of stratum corneum-piercing microprojections having a biocompatible coating formed from a coating formulation having at least one natriuretic peptide.

In other embodiments, the method of the invention comprises the steps of providing a microprojection member, placing said microprojection member inside packaging adapted to control environmental conditions, reducing moisture content inside the packaging, sealing said microprojection member with said packaging, and exposing the microprojection member to radiation selected from the group consisting of gamma radiation and e-beam, wherein the radiation is sufficient to reach a desired sterility assurance level. The microprojection member preferably includes a plurality of stratum corneum-piercing microprojections having a biocompatible coating formed from a coating formulation having at least one natriuretic peptide.

In additional embodiments, the invention is a transdermal natriuretic peptide delivery system, comprising a microprojection member including a plurality of microprojections that are adapted to pierce the stratum corneum of a patient having a biocompatible coating disposed on the microprojection member, the coating being formed from a coating formulation having at least one natriuretic peptide and packaging adapted to control environmental conditions sealed around the microprojection member, wherein the sealed package has been exposed to radiation to sterilize the microprojection member. Preferably, a desiccant is sealed inside the packaging with the microprojection member. Also preferably, the microprojection member is mounted on a pre-dried retainer ring.

In one embodiment of the invention, the packaging is purged with nitrogen.

In another embodiment, the packaging comprises a foil pouch.

In additional embodiments, the invention is a transdermal system adapted to deliver a natriuretic peptide, comprising a microprojection member including a plurality of microprojections that are adapted to pierce the stratum corneum of a patient, a hydrogel formulation having at least one natriuretic peptide in communication with the microprojection member, and packaging adapted to control environmental conditions sealed around the microprojection member, wherein the sealed package has been exposed to radiation to sterilize the microprojection member.

In other embodiments, the invention is a transdermal system adapted to deliver a natriuretic peptide, comprising a microprojection member including a plurality of microprojections that are adapted to pierce the stratum corneum of a patient, a solid state formulation having at least one natriuretic peptide disposed proximate to the microprojection member, and packaging adapted to control environmental conditions sealed around the microprojection member, wherein the sealed package has been exposed to radiation to sterilize the microprojection member.

In one embodiment, the solid state formulation is a solid film made by casting a liquid formulation comprising at least one natriuretic peptide, a polymeric material, a plasticizing agent, a surfactant and a volatile solvent.

In another embodiment of the invention, the solid state formulation is formed by a spray drying process, a freeze drying process, a spray freeze drying process or a supercritical fluid process.

In one embodiment of the invention, the microprojection member has a microprojection density of at least approximately 10 microprojections/cm², more preferably, in the range of at least approximately 200 -2000 microprojections/cm².

In one embodiment, the microprojection member is constructed out of stainless steel, titanium, nickel titanium alloys, or similar biocompatible materials.

In another embodiment, the microprojection member is constructed out of a non-conductive material, such as polymeric materials.

Alternatively, the microprojection member can be coated with a non-conductive material, such as Parylene®, or a hydrophobic material, such as Teflon®, silicon or other low energy material.

The coating formulations applied to the microprojection member to form solid biocompatible coatings can comprise aqueous and non-aqueous formulations. In at least one embodiment of the invention, the formulation(s) includes at least one natriuretic peptide, which can be dissolved within a biocompatible carrier or suspended within the carrier.

Preferably, the natriuretic peptide is selected from the family comprising atrial natriuretic peptides (ANP), B-type or brain natriuretic peptides (BNP), C-type natriuretic peptides (CNP) and urodilatins, and analogs, active fragments, degradation products, salts, variants, simple derivatives and combinations thereof. In a preferred embodiment, the natriuretic peptide comprises a B-type natriuretic peptide (BNP), more preferably, hBNP (1-32).

In one embodiment of the invention, the natriuretic peptide comprises in the range of approximately 1-40 wt. % of the coating formulation.

In one embodiment, the amount of the natriuretic peptide contained in the coating formulation is in the range of approximately 1-2000 μg.

Preferably, the dose of natriuretic peptide delivered transdermally via the aforementioned natriuretic peptide methods is in the range of approximately 10-2000 μg/day, more preferably, in the range of approximately 10-1000 μg/day.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection member;

FIG. 2 is a perspective view of the microprojection member shown in FIG. 1 having a coating deposited on the microprojections, according to the invention;

FIG. 3 is a side sectional view of a retainer having a microprojection member disposed therein, according to the invention;

FIG. 4 is a perspective view of the retainer shown in FIG. 7;

FIG. 5 is a graph illustrating total purity of nesiritide at varying gamma irradiation levels and temperatures, according to the invention;

FIG. 6 is a graph illustrating degradation products of nesiritide at varying gamma irradiation levels and temperatures, according to the invention;

FIG. 7 is a graph illustrating total purity of irradiated nesiritide treated with antioxidants at varying temperatures, according to the invention;

FIG. 8 is a graph illustrating degradation products of irradiated nesiritide treated with antioxidants at varying temperatures, according to the invention;

FIG. 9 is a graph illustrating total purity of nesiritide irradiated under selected environmental conditions, according to the invention;

FIG. 10 is a graph illustrating degradation products of nesiritide irradiated under selected environmental conditions, according to the invention;

FIG. 11 is a graph illustrating a degradation product of nesiritide gamma irradiated under selected environmental conditions at varying temperatures, according to the invention;

FIG. 12 is a graph illustrating other degradation products of nesiritide gamma irradiated under selected environmental conditions at varying temperatures, according to the invention;

FIG. 13 is a graph illustrating the total purity of nesiritide irradiated by e-beam at varying temperatures, according to the invention;

FIG. 14 is a graph illustrating degradation products of nesiritide irradiated by e-beam at varying temperatures, according to the invention;

FIG. 15 is a graph illustrating another degradation product of nesiritide irradiated by e-beam at varying temperatures, according to the invention;

FIG. 16 is a graph illustrating other degradation products of nesiritide irradiated by e-beam at varying temperatures, according to the invention;

FIG. 17 is a graph illustrating the total purity of gamma irradiated nesiritide sterilized under selected environmental conditions at varying temperatures, according to the invention;

FIG. 18 is a graph illustrating degradation products of gamma irradiated nesiritide sterilized under selected environmental conditions at varying temperatures, according to the invention;

FIG. 19 is a graph illustrating another degradation product of gamma irradiated nesiritide sterilized under selected environmental conditions at varying temperatures, according to the invention;

FIG. 20 is a graph illustrating other degradation products of gamma irradiated nesiritide sterilized under selected environmental conditions at varying temperatures, according to the invention;

FIG. 21 is a graph illustrating the total purity of e-beam irradiated nesiritide sterilized under selected environmental conditions at varying temperatures, according to the invention;

FIG. 22 is a graph illustrating degradation products of e-beam irradiated nesiritide sterilized under selected environmental conditions at varying temperatures, according to the invention;

FIG. 23 is a graph illustrating another degradation product of e-beam irradiated nesiritide sterilized under selected environmental conditions at varying temperatures, according to the invention;

FIG. 24 is a graph illustrating other degradation products of e-beam irradiated nesiritide sterilized under selected environmental conditions at varying temperatures, according to the invention;

FIG. 25 is a graph illustrating the total purity of gamma and e-beam irradiated nesiritide sterilized under selected environmental conditions at varying temperatures, according to the invention; and

FIG. 26 is a graph illustrating the degradation profile of gamma and e-beam irradiated nesiritide sterilized under selected environmental conditions at varying temperatures, according to the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a peptide” includes two or more such peptides; reference to “a microprojection” includes two or more such microprojections and the like.

### Definitions

The term “transdermal”, as used herein, means the delivery of an agent into and/or through the skin for local or systemic therapy. The term “transdermal” thus means and includes intracutaneous, intradermal and intraepidermal delivery of an agent, such as a peptide, into and/or through the skin via passive diffusion as well as energy-based diffusional delivery, such as iontophoresis and phonophoresis.

The term “transdermal flux”, as used herein, means the rate of transdermal delivery.

The term “natriuretic peptide”, as used herein, thus means a peptide that exhibits natriuretic activity. The term “natriuretic peptide” thus includes atrial natriuretic peptides (ANP), brain or B-type natriuretic peptides (BNP), C-type natriuretic peptides (CNP), urodilatins and peptides analogous thereto, and analogs, active fragments, degradation products, salts, variants, derivatives and combinations thereof.

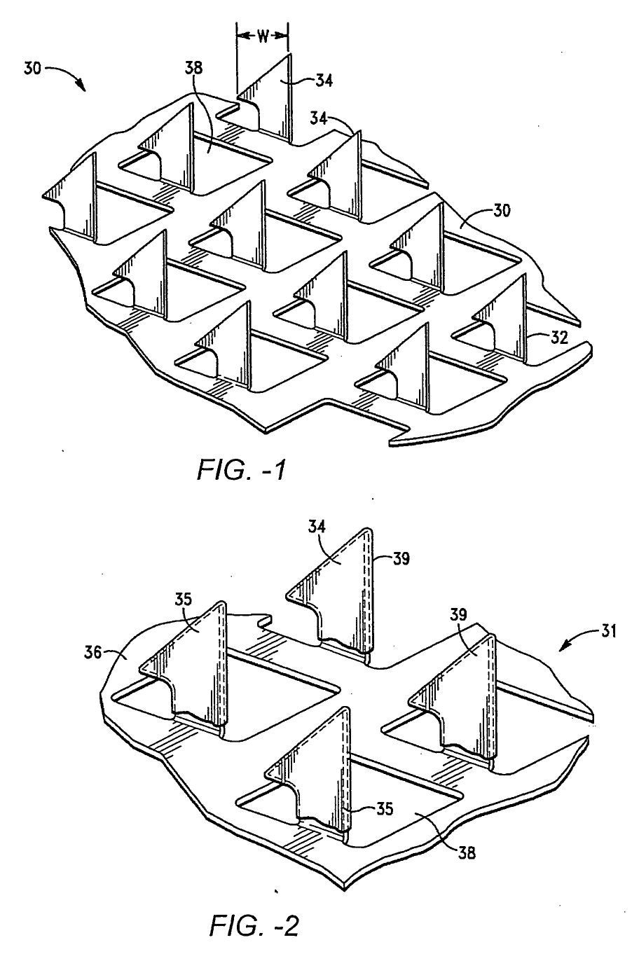

The term “brain natriuretic peptide (BNP)”, as used herein, refers to an amino acid sequence that is encoded by a DNA capable of hybridizing to an effective portion of the DNA shown in FIG. 1 of U.S. Pat. No. 5,674,710 and which has natriuretic activity.

The terms “Nesiritide” and “hBNP”, as used herein, refer to a recombinant form of human B-type natriuretic peptide, peptides analogous thereto and active fragments thereof. The terms thus include, without limitation, hBNP(1-32).

The term “co-delivering”, as used herein, means that a supplemental agent(s) is administered transdermally either before the natriuretic peptide is delivered, before and during transdermal flux of the natriuretic peptide, during transdermal flux of the natriuretic peptide, during and after transdermal flux of the natriuretic peptide, and/or after transdermal flux of the natriuretic peptide. Additionally, two or more natriuretic peptides may be formulated in the coatings and/or hydrogel formulation, resulting in co-delivery of the natriuretic peptides.

It is to be understood that more than one natriuretic peptide can be incorporated into the agent source, formulations, and/or coatings and/or solid state formulations of this invention, and that the use of the term “natriuretic peptide” in no way excludes the use of two or more such peptides.

The term “microprojections” or “microprotrusions”, as used herein, refers to piercing elements which are adapted to pierce or cut through the stratum corneum into the underlying epidermis layer, or epidermis and dermis layers, of the skin of a living animal, particularly, a mammal and, more particularly, a human.

In one embodiment of the invention, the piercing elements have a projection length less than 1000 microns. In a further embodiment, the piercing elements have a projection length of less than 500 microns, more preferably, less than 250 microns. The microprojections further have a width (designated “W” in FIG. 1) in the range of approximately 25-500 microns and a thickness in the range of approximately 10-100 microns. The microprojections may be formed in different shapes, such as needles, blades, pins, punches, and combinations thereof.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 1. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “coating formulation”, as used herein, is meant to mean and include a freely flowing composition or mixture that is employed to coat the microprojections and/or arrays thereof. The natriuretic peptide, if disposed therein, can be in solution or suspension in the formulation.

The term “biocompatible coating” and “solid coating”, as used herein, is meant to mean and include a “coating formulation” in a substantially solid form.

The term “solid state formulation”, as used herein, is meant to mean and include solid films formed by casting, and powders or cakes formed by spray drying, freeze drying, spray freeze drying and supercritical fluid extraction.

As indicated above, the present invention generally comprises a method for sterilizing a transdermal delivery system at the end of the manufacturing process. The invention also comprises the sterilized delivery systems. The transdermal delivery system includes a microprojection member (or system) having a plurality of microprojections (or array thereof) that are adapted to pierce through the stratum corneum into the underlying epidermis layer, or epidermis and dermis layers. The microprojection-member (or system) also includes at least one source or delivery medium of natriuretic peptide (i.e., biocompatible coating, hydrogel formulation and solid state formulation). The transdermal delivery system is terminally sterilized by exposure to sufficient radiation to achieve a desired sterility assurance level.

Gamma radiation can be delivered by conventional methods, such as by using Cobalt-60 as a radiation source. As one having skill in the art will recognize, a commercial Cobalt-60 sterilizer yields a rate of irradiation in the range of approximately 0.3 Gy/hr and 9.6 kGy/hr. Americium-241 can also be used, and generally irradiate at a rate of approximately 0.3 mGy/hr. Other isotopes can also be used to deliver gamma radiation at a desired rate. E-beam radiation is conventionally generated at substantially higher rates than gamma radiation, such as approximately 100 kGy/hr. In preferred embodiments, the dose rate is 3.0 kGy/hr or greater to minimize the processing time required to achieve a dose sufficient to reach the desired level of sterility.

The radiation dose required for terminal sterilization can be determined by conventional methods. For example, the dose requirements to achieve a sterility assurance level (SAL) of 10⁻⁶ can be assessed from microbiological and manufacturing considerations. In one embodiment, a low dose is based on zero bioburden (8.2kGy using ISO 11137 Method 2B) plus one augmentation (15 kGy) for a sterility failure during the quarterly dose audit. By adding a process capability of +10%, these calculations yield a dose of 16.5 kGy.

Thus, terminal sterilization of the microprojection member loaded with natriuretic peptide is achieved by irradiating the system with e-beam or gamma irradiation. Suitable doses are in the range of approximately 10 kGy to 25 kGy kGy. Preferably, the dose is at least approximately 14 kGy. More preferably, the dose is approximately 16.5 kGy. A dose of approximately 21 kGy can also be used according to the invention.

In further embodiments of the invention, the microprojection member is mounted on a retainer ring for use with an applicator.

The system can also include packaging adapted to facilitate terminal sterilization of the microprojection member.

In the noted embodiments, it is preferable to maintain an inert, low moisture atmosphere around the microprojection member. Accordingly, the retainer ring is preferably dried prior to assembly. It is also preferable to include a desiccant in the package. Suitable desiccants include 4 Å (Angstrom) molecular sieves, 3 Å molecular sieves and silica gels.

Further, the package containing the microprojection member is preferably purged with an inert gas, such as nitrogen. In alternative embodiments, the package can be evacuated to help minimize degradation of the natriuretic peptide. In a further embodiment, the amount of oxygen in the packaging is reduced to minimize oxidative degradation.

Yet other embodiments of the invention include an antioxidant to help stabilize the natriuretic peptide during irradiation. Suitable antioxidants comprise methionine, ascorbic acid and the like. Preferably, the antioxidant is added in an amount in the range of approximately 1-5%. More preferably, the antioxidant amount is approximately 3%.

In further embodiments of the invention, irradiation of the microprojection member is conducted at reduced temperatures to stabilize the natriuretic peptide. In one embodiment, the microprojection member is irradiated at a temperature in the range of approximately −78.5 to 25° C. The microprojection members can be irradiated at a temperature of −78.5° C. under dry ice conditions. In another embodiment, the microprojection member is irradiated at a temperature in the range of approximately 0-25° C. In another embodiment, the microprojection member is irradiated at an ambient temperature in the range of approximately 20-25° C.

Additional information regarding the terminal sterilization of other biologically active agent can be found in co-pending U.S. Application Ser. Nos. 60/687,519, filed Jun. 2, 2005, and 60/687,636, filed Jun. 2, 2005, which are hereby incorporated by reference in their entirety.

Referring now to FIGS. 1 and 2, there is shown one embodiment of a microprojection member 30 for use with the present invention. As illustrated in FIG. 1, the microprojection member 30 includes a microprojection array 32 having a plurality of microprojections 34. The microprojections 34 preferably extend at substantially a 90° angle from the sheet, which in the noted embodiment includes openings 38. In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm , more preferably, in the range of at least approximately 200-2000 microprojections/cm². Preferably, the number of openings per unit area through which the agent passes is at least approximately 10 openings/cm and less than about 2000 openings/cm².

As indicated, the microprojections 34 preferably have a projection length less than of 1000 microns. In one embodiment, the microprojections 34 have a projection length of less than of 500 microns, more preferably, less than of 250 microns. The microprojections 34 also preferably have a width in the range of approximately 25-500 microns and thickness in the range of approximately 10-100 microns.

To enhance the biocompatibility of the microprojection member 30 (e.g., to minimize bleeding and irritation following application to the skin of a subject), in a further embodiment, the microprojections 34 preferably have a length less than 145 μm, more preferably, in the range of approximately 50-145 μm, even more preferably, in the range of approximately 70-140 μm. Further, the microprojection member 30 comprises an array preferably having a microprojection density greater than of 100 microprojections/cm², more preferably, in the range of approximately 200-3000 microprojections/cm².

The microprojection member 30 can be manufactured from various metals, such as stainless steel, titanium, nickel titanium alloys, or similar biocompatible materials.

According to the invention, the microprojection member 30 can also be constructed out of a non-conductive material, such as a polymer.

Alternatively, the microprojection member can be coated with a non-conductive material, such as Parylene®, or a hydrophobic material, such as Teflon®, silicon or other low energy material. The noted hydrophobic materials and associated base (e.g., photoreist) layers are set forth in U.S. application Ser. No. 60/484,142, which is incorporated by reference herein.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

According to the invention, the natriuretic peptide to be administered to a host can be contained in a biocompatible coating that is disposed on the microprojection member 30 or contained in a hydrogel formulation or contained in both the biocompatible coating and the hydrogel formulation. Preferably, the hydrogel formulations of the invention comprise water-based hydrogels. Hydrogels are preferred formulations because of their high water content and biocompatibility. Also preferably, the hydrogel is configured as a gel pack.

In a further embodiment, wherein the microprojection member includes an agent-containing solid state formulation, the natriuretic peptide can be contained in the biocompatible coating, hydrogel formulation or solid state formulation, or in all three delivery mediums.

In one embodiment, the solid state formulation is a solid film made by casting a liquid formulation comprising at least one natriuretic peptide, a polymeric material, such as hyroxyethyl starch, dextran, hydroxyethylcellulose (HEC), hydroxypropylmethylcellulose (HPMC), hydroxypropycellulose (HPC), methylcellulose (MC), hydroxyethylmethylcellulose (HEMC), ethylhydroxethylcellulose (EHEC), carboxymethylcellulose (CMC), poly(vinyl alcohol), poly(ethylene oxide), poly(2-hydroxyethymethacrylate), poly(n-vinyl pyrolidone) and pluronics, a plasticizing agent, such as glycerol, propylene glycol and polyethylene glycol, a surfactant, such as Tween 20 and Tween 80, and a volatile solvent, such as water, isopropanol, methanol and ethanol.

In one embodiment, the liquid formulation used to produce the solid film comprises: 0.1-20 wt. % natriuretic peptide, 5-40 wt. % polymer, 5-40 wt. % plasticizer, 0-2 wt. % surfactant, and the balance of volatile solvent.

Following casting and subsequent evaporation of the solvent, a solid film is produced.

Preferably, the natriuretic peptide is present in the liquid formulation used to produce the solid film at a concentration in the range of approximately 0.1-2 wt. %.

In another embodiment of the invention, the solid state formulation is a powder or cake formulation. Suitable formulations are achieved by spray drying, freeze drying, spray freeze drying and supercritical fluid processing. According to the invention, these methods form a high payload powder or cake solid state formulation that is reconstituted by the hydrogel formulation prior to the transdermal delivery of the natriuretic peptide. Preferably, the powder formulations are adapted to have relatively high porosity to facilitate reconstitution and improve patient compliance.

The noted processes of making powder and cake formulations are highly efficient, typically having yields of approximately 85%. Further, the processes do not require the use of plasticizers that depress Tg and, correspondingly, can reduce shelf life. Preferably, the formulations subjected to drying or supercritical fluid extraction in the noted methods also comprise a carbohydrate, such as a saccharide or a sugar alcohol to help protect the natriuretic peptide. Also preferably, the formulation includes an antioxidant, such as methionine. Specific formulations are discussed below.

Spray drying, freeze drying, spray freeze drying and supercritical fluid extraction afford good control over particle size and distribution, particle shape and morphology. The noted techniques are also known in the art. For example, the spray freeze drying process is ideal for high valued therapeutic drugs as batch sizes as small as 300 mg can be produced with high yields.

As can be appreciated, the spray drying, freeze drying, spray freeze drying and supercritical fluid extraction processes generate a cake form which is readily incorporated into the microprojection system discussed above. Alternatively, the processes generate a powder form, which is further processed to form a cake. In other embodiments, the powder form is held in a container adapted to communicate with the hydrogel. Preferably, such embodiments include strippable release liners to separate the powder form from the hydrogel until reconstitution is desired.

In one embodiment of the invention, a suitable spray freeze drying process generally involves exposing an atomized liquid formulation containing the natriuretic peptide to liquid nitrogen. Under the reduced temperature, the atomized droplets freeze in a time-scale of milliseconds. This freezing process generates very fine ice crystals, which are subsequently lyophilized. The noted technique generates a powder having a high intraparticle porosity, allowing rapid reconstitution in aqueous media. Examples of suitable nesiritide formulations are given below.

In another embodiment of the invention, a suitable supercritical fluid process generally involves crystallizing a liquid formulation of the natriuretic peptide in a solvent that is maintained above its critical temperature and pressure. Controlling the conditions of the crystallization process allows the production of a natriuretic peptide powder having desired particle size and distribution, particle shape and morphology.

According to the invention, at least one natriuretic peptide is contained in at least one of the aforementioned delivery mediums. The amount of the natriuretic peptide that is employed in the delivery medium and, hence, microprojection system will be that amount necessary to deliver a therapeutically effective amount of the natriuretic peptide to achieve the desired result. In practice, this will vary widely depending upon the particular natriuretic peptide, the site of delivery, the severity of the condition, and the desired therapeutic effect.

In one embodiment, the microprojection member includes a biocompatible coating that contains at least one natriuretic peptide, preferably, hBNP(1-32). The microprojection member is terminally sterilized to a desired sterility assurance level. Upon piercing the stratum comeum layer of the skin, the peptide-containing coating is dissolved by body fluid (intracellular fluids and extracellular fluids such as interstitial fluid) and released into the skin (i.e., bolus delivery) for systemic therapy. Preferably, the total dose of natriuretic peptide delivered transdermally is in the range of approximately 10-2000 μg/day, more preferably, 10-1000 μg/day.

Referring now to FIG. 2, there is shown a microprojection member 31 having microprojections 34 that include a biocompatible coating 35 of the natriuretic peptide. According to the invention, the coating 35 can partially or completely cover each microprojection 34. For example, the coating 35 can be in a dry pattern coating on the microprojections 34. The coating 35 can also be applied before or after the microprojections 34 are formed. Additional information regarding the use of a transdermal natriuretic peptide delivery system can be found in co-pending U.S. Application Ser. No. 60/600,638, filed Aug. 10, 2004, which is hereby incorporated by reference in its entirety.

According to the invention, the coating 35 can be applied to the microprojections 34 by a variety of known methods. Preferably, the coating is only applied to those portions the microprojection member 31 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 34 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

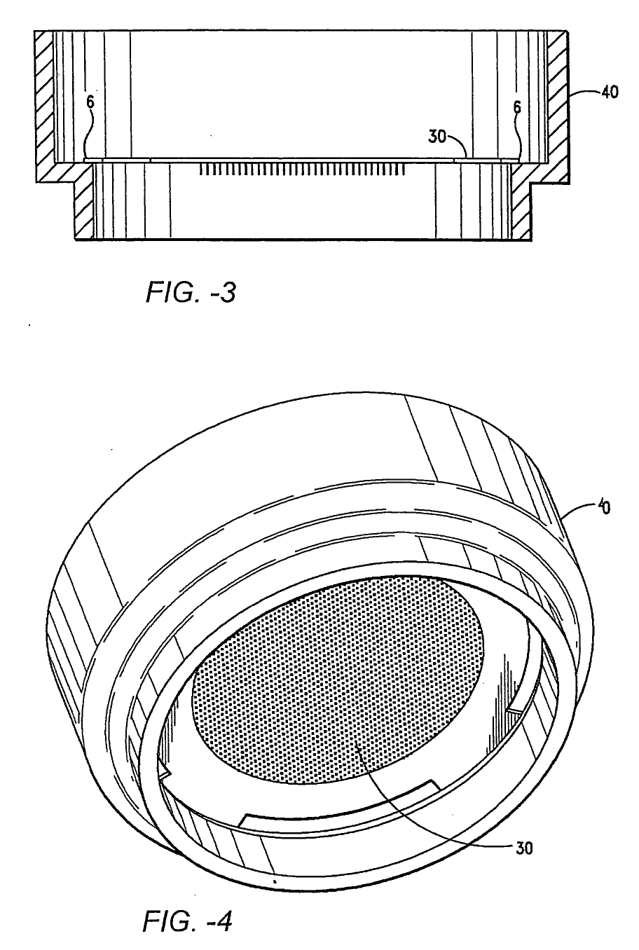

Referring now to FIGS. 3 and 4, for storage and application, the microprojection member 30 is preferably suspended in a retainer ring 40 by adhesive tabs 6, as described in detail in U.S. application Ser. No. 09/976,762 (Pub. No. 2002/0091357), which is incorporated by reference herein in its entirety.

After placement of the microprojection member in the retainer ring 40, the microprojection member is applied to the patient's skin. Preferably, the microprojection member is applied to the patient's skin using an impact applicator, as described in Co-Pending U.S. application Ser. No. 09/976,978, which is incorporated by reference herein in its entirety. As discussed above, retainer ring 40 is preferably pre-dried prior to packaging to reduce the amount of moisture in the atmosphere surrounding the microprojection member during irradiation.

As indicated, according to one embodiment of the invention, the coating formulations applied to the microprojection member 30 to form solid biocompatible coatings can comprise aqueous and non-aqueous formulations having at least one natriuretic peptide. According to the invention, the natriuretic peptide can be dissolved within a biocompatible carrier or suspended within the carrier.

In a preferred embodiment, the brain natriuretic peptide comprises a human B-type natriuretic peptide (BNP), including hBNP(1-32) and analogs, salts, variants, active fragments and simple derivatives thereof. In a preferred embodiment, the coating formulation comprises a 4:1 formulation of sucrose:BNP. The amount and type of adjuvant is adapted to optimize the stability of the natriuretic peptide during sterilization.

In one embodiment of the invention, the natriuretic peptide comprises in the range of approximately 1-40 wt. % of the coating formulation.

In one embodiment, the amount of the natriuretic peptide contained in the coating formulation is preferably in the range of approximately 1-2000 μg.

Preferably, the coating formulations have a viscosity less than approximately 500 centipoise and greater than 3 centipose.

In one embodiment of the invention, the coating thickness is less than 25 microns, more preferably, less than of 10 microns as measured from the microprojection surface.

The desired coating thickness is dependent upon several factors, including the required dosage and, hence, coating thickness necessary to deliver the dosage, the density of the microprojections per unit area of the sheet, the viscosity and concentration of the coating composition and the coating method chosen. The thickness of coating 35 applied to microprojections 34 can also be adapted to optimize stability of the natriuretic peptide.

In all cases, after a coating has been applied, the coating formulation is dried onto the microprojections 34 by various means. In a preferred embodiment of the invention, the coated microprojection member 30 is dried in ambient room conditions. However, various temperatures and humidity levels can be used to dry the coating formulation onto the microprojections. Additionally, the coated member can be heated, stored under vacuum or over desiccant, lyophilized, freeze dried or similar techniques used to remove the residual water from the coating.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169,383, the disclosures of which are incorporated by reference herein in their entirety.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may by attained in various manners.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods, such as sonophoresis or piezoelectric devices, can be used in conjunction with the invention.

### EXAMPLES

The following examples are given to enable those skilled in the art to more clearly understand and practice the present invention. They should not be considered as limiting the scope of the invention but merely as being illustrated as representative thereof.

### Example 1

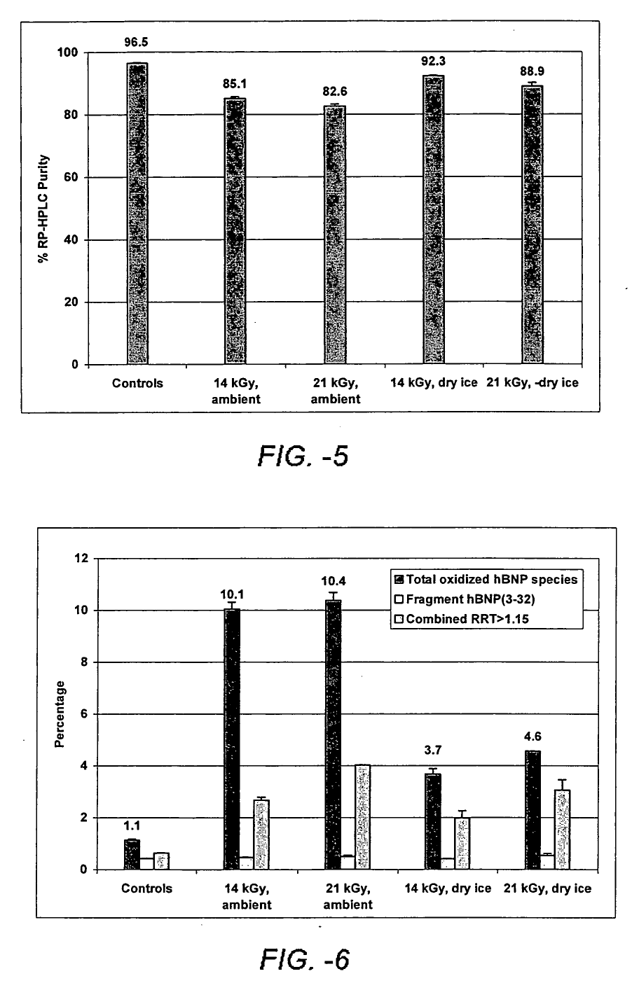

A formulation comprising 4:1 sucrose:BNP was coated onto MF1035 microprojection members (Macroflux®, available from Alza Corporation, Mountain View, Calif.). The microprojection members were packaged with a nitrogen purge and subjected to gamma irradiation doses of 14 and 21 kGy under dry ice and ambient temperatures. The total purity results are shown in FIG. 5. FIG. 6 summarizes the degradation product profile (as measured by a validated reverse phase, high-pressure liquid chromatography “RP-HPLC” method). This example shows that samples irradiated at 14 kGy under dry ice lost only 4.2% total purity. The loss in purity was due mainly to increased oxidation as shown by the RP-HPLC chromatogram. Several small degradents (<0.1%) were detected at higher retention time relative to the main BNP peak (RRT>1.25). These peaks can be attributed to acetate modifications.

### Example 2

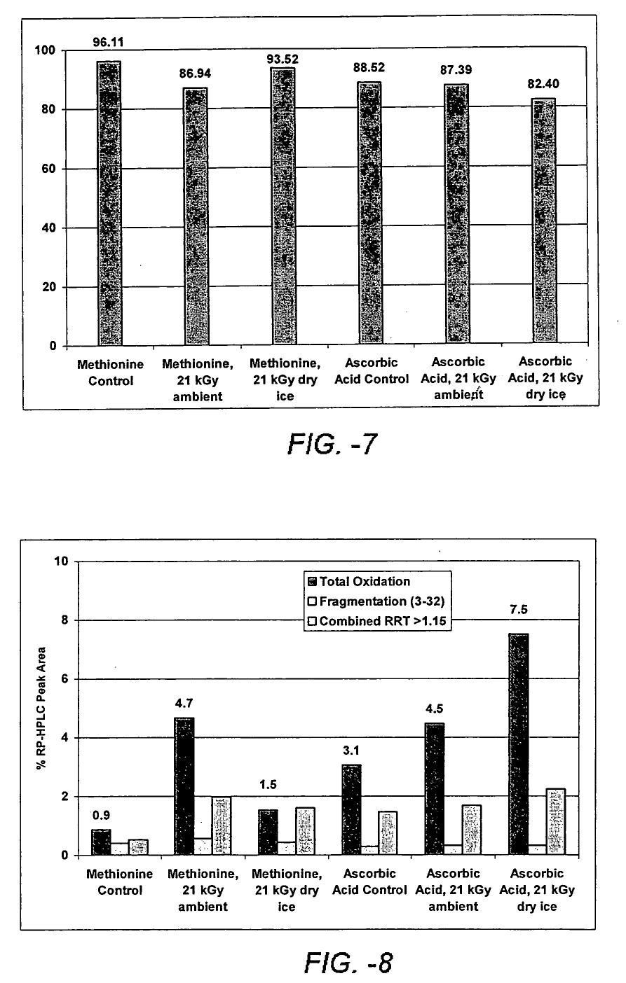

The addition of an antioxidant to the formulation can mitigate degradation caused by irradiation. In this example, 3% by weight of selected antioxidants were added to 4:1 sucrose:BNP formulations. As shown in FIG. 7, the addition of methionine obtained from Sigma (St. Louis, Mo) provides a greater degree of protection than ascorbic acid. These results indicate that the addition of methionine improves the stability of the coated formulation during gamma irradiation, and to a greater extent at a low temperature (dry ice). Samples formulated with methionine irradiated with 21 kGy under dry ice only lost 2.6% purity relative to non-irradiated controls. Methionine oxidation at position 4 and 15 were the major degradation components as shown in FIG. 8. However, the combined degradation peaks with high retention times (RRT>1.25) are substantial even though all individual peaks in this region are below 0.1% of the total peak area.

### Example 3

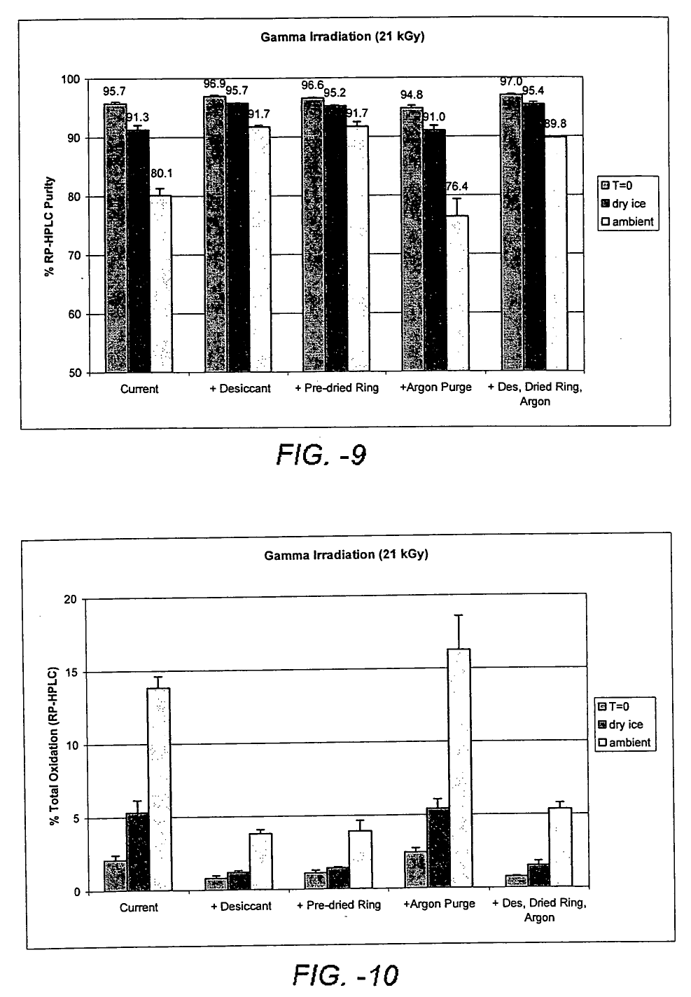

As shown in FIG. 9, the column labeled “Current” indicates that irradiation of hBNP systems packaged with a nitrogen purge lost approximately 10% of the initial drug purity following an irradiation dose of 21 kGy at an ambient temperature. Further, adjusting the irradiation temperature under dry ice minimized the loss to approximately 5%. Additional protection of the hBNP was obtained by providing a dry packaging environment. FIG. 9 compares the stability of hBNP following gamma irradiation for samples containing either a desiccant or a pre-dried retainer rings, or substituting an argon purge for the nitrogen purge. These results indicate the loss in total purity was reduced to only approximately 1% under dry ice and approximately 4% at an ambient temperature at the high irradiation dose of 21 kGy, using either the desiccant or the pre-dried ring.

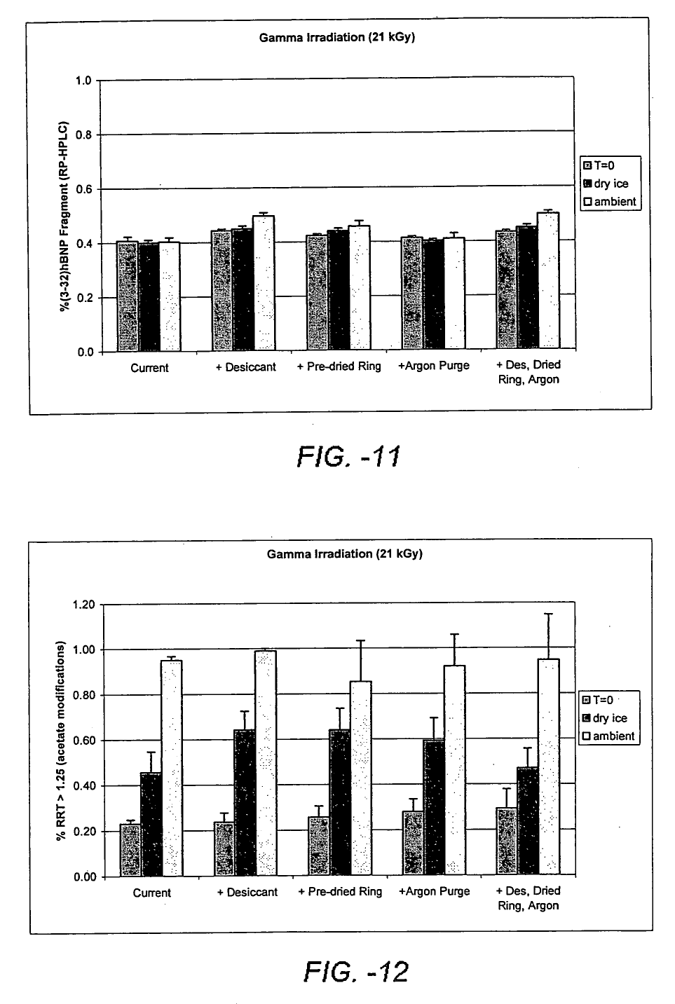

FIGS. 10-12 show analyses of the degradation products under the noted conditions. Specifically, FIG. 10 shows the species attributable to methionine oxidation, FIG. 11 shows the species corresponding to fragmentation of BNP, and FIG. 12 shows the oxidation species having relative retention times greater than 1.25.

This example also shows that packaging containing a desiccant, which has a relatively high oxygen content, has substantially equivalent product stability following terminal sterilization process as packaging containing a pre-dried retainer ring, which has a significantly lower amount of oxygen in the head space. Accordingly, moisture vapor is apparently a greater contributor to product instability during terminal sterilization than free oxygen.

FIGS. 9-12 also show that the use of argon as a purge gas does not provide as great a degree of protection as nitrogen even though the head space analysis of packaging purged with nitrogen or argon appeared to be similar. Conventionally, argon would be expected to be a more inert gas than nitrogen. Combining an argon purge with the use of a desiccant and a pre-dried ring offers better protection than the nitrogen control and the argon purged systems, but not as well as the desiccant or pre-dried ring systems. These results indicate reduced packaging humidity produces a positive contribution to product stability, but the argon purge has an apparent negative contribution in these samples.

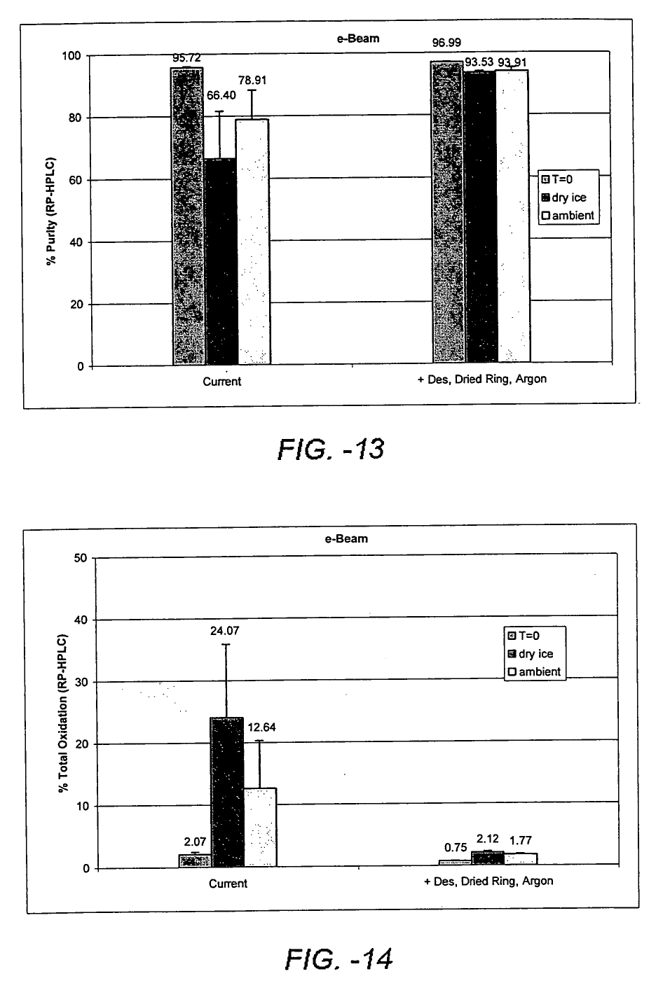

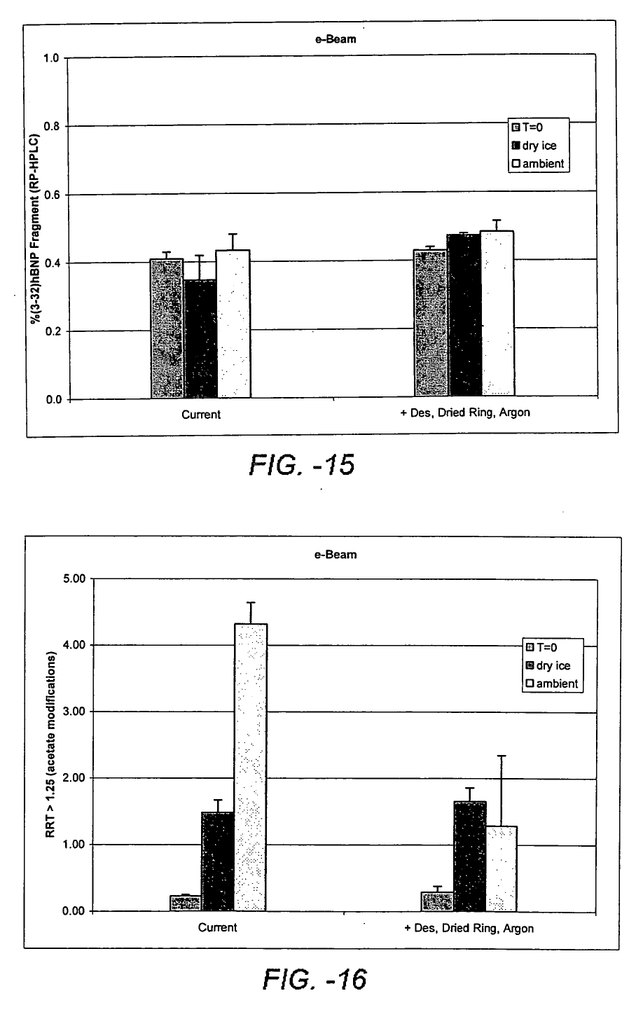

A comparison of e-beam irradiation to gamma irradiation under similar conditions is shown in FIGS. 13-16. The total purity results indicates that samples treated by e-beam have improved stability relative to gamma irradiated samples at the same temperatures, as shown in FIG. 13. FIGS. 14-16 detail the major degradation pathways for BNP coated on microprojections for all conditions treated by e-beam. Unexpectedly, e-beam treatment under dry ice demonstrated less protection than treatment at an ambient temperature. Further, this example demonstrates that the packaging environment has a significant effect on the performance of the e-beam treatment. All modes of degradation are reduced for the microprojection members packaged with desiccant and pre-dried retainer rings and purged with argon.

### Example 4

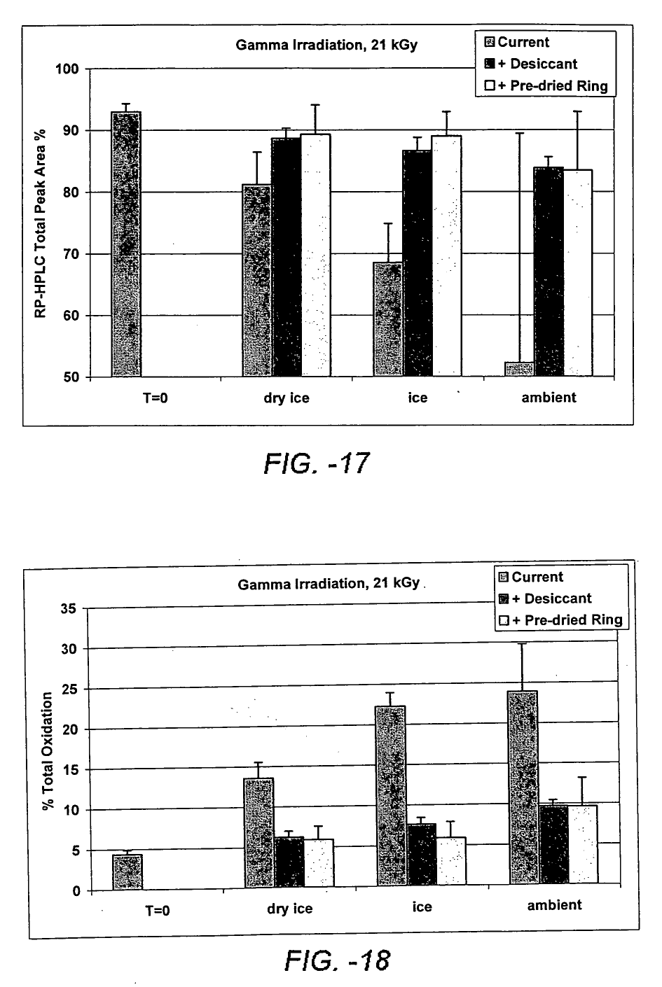

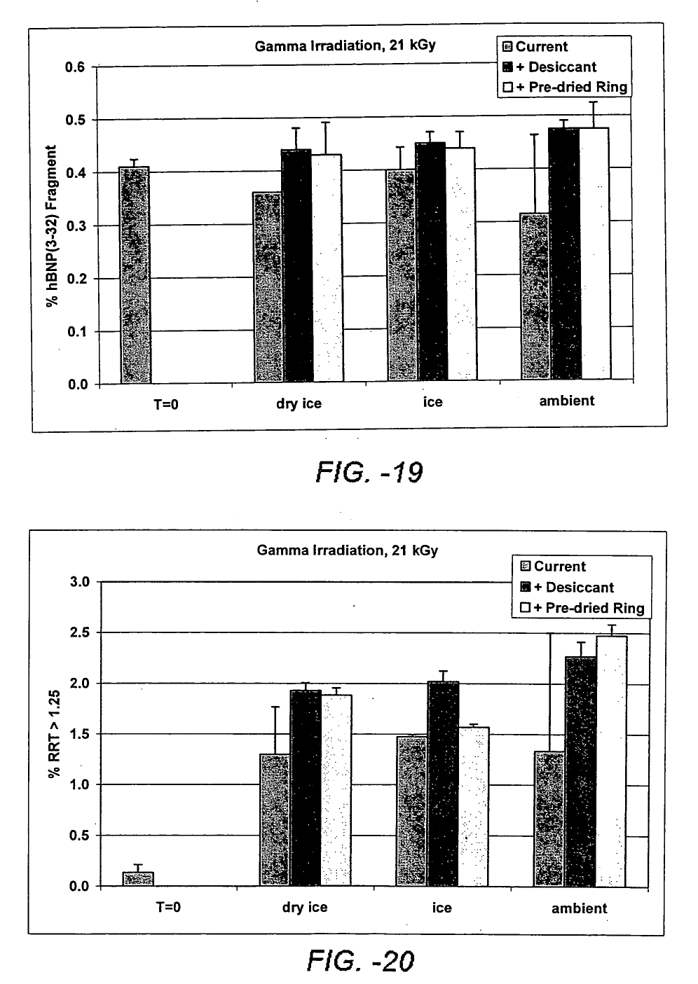

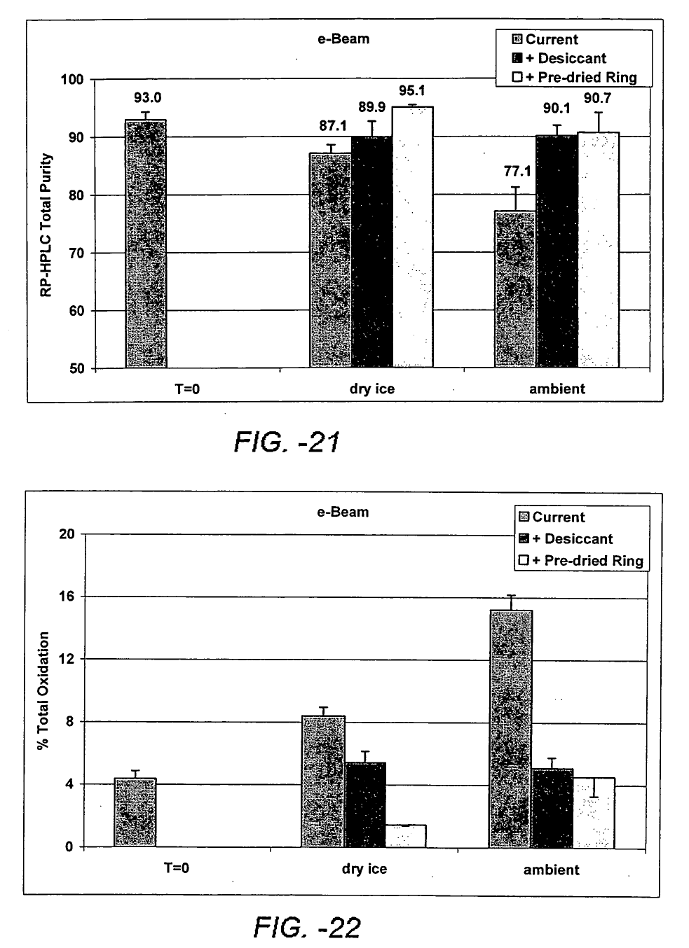

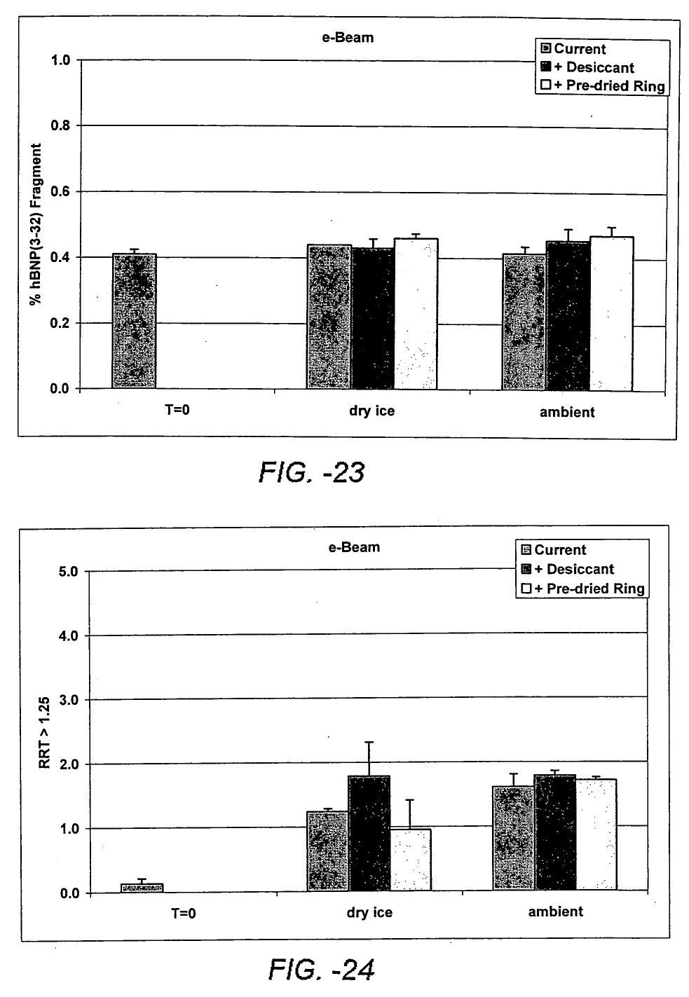

In this example, methionine from JT Baker (Phillipsburg, N.J.) was added to the BNP formulations in the amount of 1% by weight. These coating formulations were then irradiated using either e-beam or gamma radiation at varying temperatures and under varying atmospheric conditions. FIGS. 17-20 show the purity and degradation of the samples following gamma irradiation and FIGS. 21-24 show the purity and degradation of the samples following e-beam irradiation.

These results show a somewhat negative protective effect for samples with an antioxidant. However, the use of a desiccant or a pre-dried retainer ring significantly improved the stability of the product. FIGS. 18-20, in particular, show that the oxidation products and that the degradents having a high retention time were the most significant degradation products.

Turning to FIGS. 21-24, the results from this example show that e-beam irradiation caused less degradation than gamma irradiation. Further, systems packaged with pre-dried retainer rings performed better than systems stored with desiccant when terminally sterilized by e-beam. The best performing system retained 95.1% of the BNP purity, representing only approximately a 2% drop from the initial material purity. Again, additional packaging measures to reduce residual moisture contribute significantly to preserving the purity of the BNP through e-beam treatment.

### Example 5

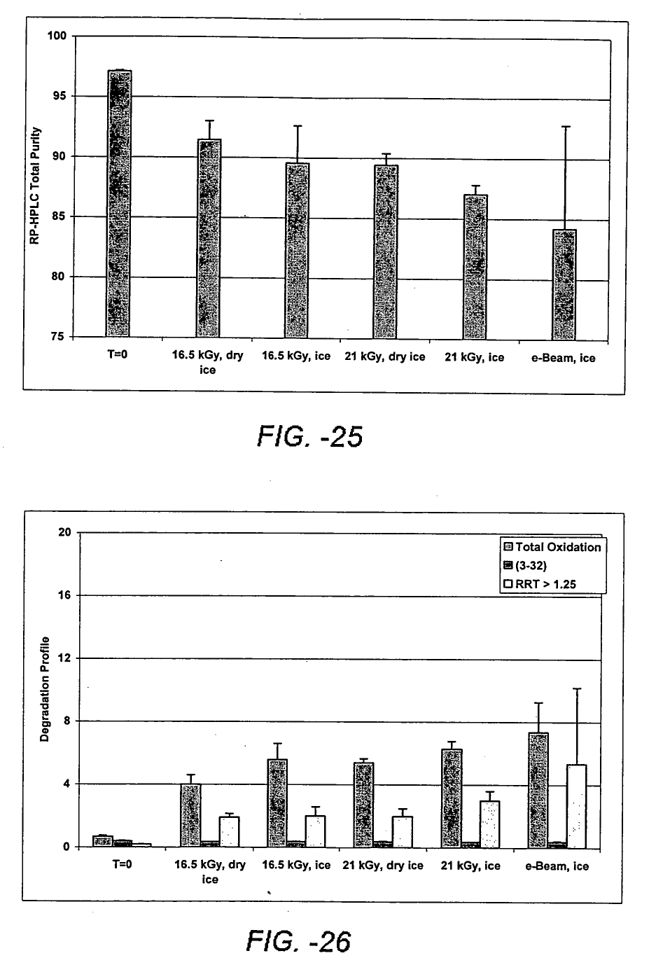

Microprojection members were coated with the 4:1 formulation containing 1% by weight of methionine from Sigma and packaged with desiccant in this example. The purity results following terminal sterilization are shown in FIG. 25. At the same irradiation temperature and dose (21 kGy), microprojection members having a coating formulation with 1% Sigma methionine lost an additional 5% of purity due to increased oxidation as shown in FIG. 26 as compared to microprojection members having a coating formulation formulated with 3% Sigma methionine as shown in FIG. 8.

### Example 6

Five hBNP formulations were prepared by freeze drying and spray freeze drying processes to assess reconstitution time. In each case the reconstitution medium was deionised water and the amount added to each formulation was such that the resulting concentration of hBNP was 100 mg/ml. The hBNP spray freeze dried powder or freeze dried cake was allowed to dissolve without the aid of agitation after addition of deionised water to the powder hBNP formulations. The reconstitution results are shown in Table 1.

TABLE 1
Reconstitution times of solid state hBNP formulations

| | | | Reconstitution | State after |
|---|---|---|---|---|
| Lot No. | Composition | Process | time (min) | reconstitution |
| 8269166A | 49% w/w hBNP, 49% w/w | SFD | 1 | Liquid |
| | sucrose, 2% methionine (50% | | | |
| | solids content). | | | |
| 8269166B | 49% w/w hBNP, 49% w/w | SFD | 1 | Liquid |
| | sucrose, 2% methionine (30% solids | | | |
| | content). | | | |
| 8269170A | 5.1% w/w hBNP, 5.1% w/w | FD | 1.5 | Liquid |
| | sucrose, 1.3% w/w mannitol, | | | |
| | 0.2% w/w methionine. | | | |
| 8269170B | 5.0% w/w hBNP, 5.0% w/w | FD | 1.5 | Liquid |
| | sucrose, 2.5% w/w mannitol, | | | |
| | 0.2% w/w methionine. | | | |
| 8269170C | 5.1% w/w hBNP, 2.6% w/w | FD | 1.5 | Liquid |
| | sucrose, 2.6% w/w mannitol, | | | |
| | 0.2% w/w methionine. | | | |

### Example 7

In this example, the storage stability of powder solid-state formulations was assessed. Three formulations were prepared and dispensed in glass vials under an inert atmosphere. The glass vials were capped and stored at an ambient temperature and 40° C. for a period of two weeks to determine stability. As shown in Table 2, the freeze dried and spray freeze dried formulation exhibit adequate stability over the storage period.

TABLE 2
Two week stability summary

| | | | T = 0 | T = 2 weeks |
|---|---|---|---|---|
| Process | Lot No | Formulation composition | hBNP Purity (%) | hBNP Purity (%) |
| FD | 8520005A | 43.5% w/w hBNP, 43.5% w/w | 98.16 | 25° C. - 97.85 |
| | | sucrose, 10.9% w/w mannitol, | | 40° C. - 96.85 |
| | | 2.0% w/w methionine | | |
| | 8520005A | 49.0% w/w hBNP, 49.0% w/w | 97.93 | 25° C. - 97.85 |
| | | sucrose, 2.0% w/w methionine | | 40° C. - 97.23 |
| SFD | 8520007 | 49% w/w hBNP, 49% w/w | 97.87 | 25° C. - 97.71 |
| | | sucrose, 2% w/w methionine | | 40° C. - 96.95 |
| | | 40% solids content) | | |

As shown by the above examples and discussion, microprojection members having a coating formulation including a natriuretic peptide such as nesiritide can be terminally sterilized by either gamma irradiation or e-beam treatment with only a minor reduction in chemical purity using the methods of the invention. Preferably, the packaging of the microprojection members is adapted to provide an inert atmosphere with relatively low humidity during the terminal sterilization process. For example, a sealed foil pouch purged with dry nitrogen and containing desiccant has a significant stabilizing effect. Also preferably, the microprojection member is mounted on a pre-dried retainer ring prior to packaging.

Further, product degradation can also be reduced during the terminal sterilization process by reducing the temperature or by reducing the sterilization dose.

The apparatus and methods of the invention can also be employed in the treatment of various ailments, including, but not limited to, STEMI (ST-Segment Elevation Myocardial Infarction), CKD (Chronic Kidney Disease), acute coronary syndromes (Class III/IV heart failure), pulmonary hypertension and pre-eclampsia.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method for terminally sterilizing a transdermal device adapted to deliver a natriuretic peptide, comprising the steps of:
providing a microprojection member having a plurality of microprojections that are adapted to pierce the stratum comeum of a patient having a biocompatible coating disposed on said microprojection member, said coating being formed from a coating formulation having at least one natriuretic peptide disposed thereon; and
exposing said microprojection member to radiation selected from the group consisting of gamma radiation and e-beam, wherein said radiation is sufficient to reach a desired sterility assurance level.

2. The method of claim 1, further comprising the step of sealing said microprojection member inside packaging adapted to control environmental conditions surrounding said microprojection member.

3. The method of claim 2, wherein said packaging comprises a foil pouch.

4. The method of claim 2, further comprising the step of sealing a desiccant inside said packaging.

5. The method of claim 2, further comprising the step of mounting said microprojection member on a pre-dried retainer ring prior to sealing said microprojection member inside said packaging.

6. The method of claim 4, further comprising the step of mounting said microprojection member on a pre-dried retainer ring prior to sealing said microprojection member inside said packaging.

7. The method of claim 2, further comprising the step of purging said packaging with an inert gas prior to sealing said microprojection member.

8. The method of claim 7, wherein said inert gas comprises nitrogen.

9. The method of claim 2, wherein said step of exposing said microprojection member to radiation occurs at approximately −78.5-25° C.

10. The method of claim 2, wherein said step of exposing said microprojection member to radiation occurs at an ambient temperature.

11. The method of claim 2, wherein said step of exposing said microprojection member to radiation comprises delivering in the range of approximately 5 to 50 kGy.

12. The method of claim 2, wherein said step of exposing said microprojection member to radiation comprises delivering approximately 7 kGy.

13. The method of claim 2, wherein said step of exposing said microprojection member to radiation comprises delivering approximately 21 kGy.

14. The method of claim 2, wherein said step of exposing said microprojection member to radiation comprises delivering radiation at a rate of greater than approximately 3.0 kGy/hr.

15. The method of claim 2, wherein said sterility assurance level is 10⁻⁶.

16. The method of claim 2, further comprising the step of adding an antioxidant to said coating formulation.

17. A method for terminally sterilizing a transdermal device adapted to deliver a natriuretic peptide, comprising the steps of:
providing a microprojection member having a plurality of microprojections that are adapted to pierce the stratum comeum of a patient having a biocompatible coating disposed on said microprojection member, said coating being formed from a coating formulation having at least one natriuretic peptide disposed thereon; sealing said microprojection member with a desiccant inside packaging purged with nitrogen and adapted to control environmental conditions surrounding said microprojection member; and
exposing said microprojection member to radiation selected from the group consisting of gamma radiation and e-beam radiation, wherein said radiation is sufficient to reach a desired sterility assurance level.

18. The method of claim 17, further comprising the step of mounting said microprojection member on a pre-dried retainer ring prior to sealing said microprojection member inside said packaging.

19. The method of claim 17, wherein said step of exposing said microprojection member to radiation comprises delivering a dose of radiation in the range of approximately 7-21 kGy.

20. The method of claim 19, wherein said step of exposing said microprojection member to radiation occurs at an ambient temperature.

21. The method of claim 17, wherein said natriuretic peptide retains at least approximately 96% of initial purity.

22. The method of claim 21, wherein said natriuretic peptide retains at least approximately 98% of initial purity.

23. A method for terminally sterilizing a transdermal device adapted to deliver a natriuretic peptide, comprising the steps of:
providing a microprojection member having a plurality of microprojections that are adapted to pierce the stratum comeum of a patient having a biocompatible coating disposed on said microprojection member, said coating being formed from a coating formulation having at least one natriuretic peptide disposed thereon;
sealing said microprojection member inside packaging purged with an inert gas and adapted to control environmental conditions surrounding said microprojection member; and
exposing said microprojection member to e-beam radiation, wherein said radiation is sufficient to reach a desired sterility assurance level.

24. A method for terminally sterilizing a transdermal device adapted to deliver a natriuretic peptide, comprising the steps of:
providing a microprojection member having a plurality of microprojections that are adapted to pierce the stratum comeum of a patient having a biocompatible coating disposed on said microprojection member, said coating being formed from a coating formulation having at least one natriuretic peptide disposed thereon;
placing said microprojection member inside packaging adapted to control environmental conditions;
reducing moisture content inside said packaging;
sealing said microprojection member with said packaging; and
exposing said microprojection member to radiation selected from the group consisting of gamma radiation and e-beam, wherein said radiation is sufficient to reach a desired sterility assurance level.

25. A transdermal system, adapted to deliver a natriuretic peptide, comprising:
a microprojection member including a plurality of microprojections that are adapted to pierce the stratum comeum of a patient having a biocompatible coating disposed on said microprojection member, said coating being formed from a coating formulation having at least one natriuretic peptide disposed thereon; and
packaging purged with an inert gas and adapted to control environmental conditions sealed around said microprojection member;
wherein said sealed package has been exposed to radiation to sterilize the microprojection member.

26. The system of claim 25, further comprising a desiccant sealed inside said packaging with said microprojection member.

27. The system of claim 25, wherein said microprojection member is mounted on a pre-dried retainer ring.

28. The system of claim 25, wherein said packaging is purged with nitrogen.

29. The system of claim 25, wherein said packaging comprises a foil pouch.

30. The system of claim 25, wherein said natriuretic peptide comprises hBNP.

31. A transdermal system, adapted to deliver a natriuretic peptide, comprising:
a microprojection member including a plurality of microprojections that are adapted to pierce the stratum comeum of a patient;
a hydrogel formulation having at least one natriuretic peptide, wherein said hydrogel formulation is in communication with said microprojection member; and
packaging purged with an inert gas and adapted to control environmental conditions sealed around said microprojection member;
wherein said sealed package has been exposed to radiation to sterilize the microprojection member.

32. A transdermal system, adapted to deliver a natriuretic peptide, comprising:
a microprojection member including a plurality of microprojections that are adapted to pierce the stratum corneum of a patient;
a solid film disposed proximate said microprojection member, wherein said solid film is made by casting a liquid formulation comprising at least natriuretic peptide, a polymeric material, a plasticizing agent, a surfactant and a volatile solvent; and
packaging purged with an inert gas and adapted to control environmental conditions sealed around said microprojection member;
wherein said sealed package has been exposed to radiation to sterilize the microprojection member.
